(19) United States
(12) Patent Application Publication
(10) Pub. No.: US 2011/0166191 A1
(43) Pub. Date: Jul. 7, 2011

(54) 3-(2-amino-ethyl)-5-(3-cyclohexyl-propylidene)-thiazolidine-2,4-dione and its derivatives as multiple signaling pathway inhibitors and for the treatment of cancer

(76) Inventors: Shijun Zhang, Richmond, VA (US); Tai Liang Guo, Glen Allen, VA (US)

(21) Appl. No.: 12/984,825

(22) Filed: Jan. 5, 2011

Related U.S. Application Data
(60) Provisional application No. 61/292,900, filed on Jan. 7, 2010.

(51) Int. Cl. A61K 31/426; C07D 277/34; A61P 35/00; C12N 5/02; C12N 9/99

(52) U.S. Cl. 514/369; 548/183; 435/375; 435/184

(57) ABSTRACT

3-(2-amino-ethyl)-5-(3-cyclohexyl-propylidene)-thiazolidine-2,4-dione and derivatives thereof are provided for use as dual inhibitors of the Raf/MEK/ERK and PI3K/Akt pathways and for use in the treatment of cancer.

### CROSS-REFERENCE TO RELATED APPLICATIONS

This application claims benefit of U.S. provisional patent 61/292,900, filed Jan. 7, 2009, the complete contents of which is hereby incorporated by reference.

### BACKGROUND OF THE INVENTION

1. Field of the Invention

The invention generally relates to 5-alkylidenethiazolidine-2,4-dione analogs and their use as anti-cancer agents. In particular, the invention provides 3-(2-amino-ethyl)-5-(3-cyclohexyl-propylidene)-thiazolidine-2,4-dione and derivatives thereof as dual inhibitors of the Raf/MEK/ERK and PI3K/Akt pathways and for use in the treatment of cancer.

2. Background of the Invention

Cancer has surpassed heart disease as the leading cause of death in the United States in people younger than 85 and it is expected that 1.53 million cases of cancer will be diagnosed in the United States in 2009, among which more than 569,490 are expected to die [1]. In addition to the human cost, more than $72 billion is spent annually (2004) on cancer treatment, acerbating problems with the overextended U.S. health care economy. While many chemotherapeutic strategies for cancer treatment have been proposed, tested and in some cases implemented in the past few decades, these diseases remain tenacious and deadly. Thus, novel treatment strategies continue to be of very high interest.

Dysregulated signaling pathways have been implicated to promote cancer cell survival and growth, in which the Raf/MEK/extracellular signal-regulated kinase (ERK) cascade and the phosphatidylinositol 3-kinase (PI3K)/Akt cascade are the best characterized. The Raf/MEK/ERK pathway is one of the evolutionarily conserved mitogen-activated protein kinase (MAPK) pathways that play critical roles in driving proliferation and preventing apoptosis [2]. Upon activation by growth factors, serum, cytokines and osmotic stresses, ERK can phosphorylate and regulate multiple substrates such as cytoskeletal proteins, kinases and transcription factors within various cellular compartments. These events in turn result in gene expression changes and alteration in cell proliferation, differentiation and survival. This pathway has received particular attention in the past 15 years as substantial evidence has shown that aberrant activation of this pathway at different levels is involved in the oncogenesis of various human cancers, especially in melanoma, breast cancers, ovarian cancers and human leukemias [3]. Numerous structurally diverse molecules have been developed by targeting the Raf/MEK/ERK pathway in search for potential medications for various human cancers and have been extensively reviewed in recent articles [4].

PI3K/Akt signaling pathway is another signaling cascade that has been implicated to be crucial in cancer development. Genomic aberrations in this pathway are prevalent compared to any other pathway in human cancers with the possible exception of the p53 and retinoblastoma pathway [5]. Upon stimulation by growth factors and cytokines, PI3K is recruited to the plasma membrane and subsequently converts phosphatidylinositol-3,4-bisphosphate (PIP2) into phosphatidylinositol-3,4-5-trisphosphate (PIP3) that will in turn recruit and activate a serine/threonine kinase Akt together with 3′-phosphoinositide-dependent kinase (PDK). Signals through this cascade regulate many fundamental cellular functions such as cell growth, proliferation, survival, apoptosis, and metabolism through a variety of downstream effectors including proapoptotic and antiapoptotic factors, mTOR, glycogen synthase kinase-3 (GSK-3), and p53, among others. Phosphatase and tensin homolog deleted on chromosome 10 (PTEN), a negative regulator of PI3K/Akt signaling by specifically dephosphorylating PIP3 has been detected to lose its activity by either genetic or epigenetic modifications in many primary and metastatic human cancers [6]. Mutations and/or activation of PI3K and Akt have been detected in various human cancers [6]. Therefore, it is logical to target this pathway to develop potential treatment agents, and indeed small molecule inhibitors including PI3K inhibitors, Akt inhibitors and mTOR inhibitors have been developed and/or approved as treatment agents for various human cancers [7].

Notably, these two signaling pathways intimately and cooperatively link with each other, rather than exclusively, to regulate apoptosis and the survival of transformed cells. Both signaling pathways can phosphorylate and regulate many common downstream effectors involved in the regulation of cell survival and apoptosis such as CREB, Bad, Bim and caspase 9, among others [8]. Accumulating evidence has strongly suggested crosstalk and the possible existence of a feedback regulation loop between these two pathways. For example, most recently, studies have demonstrated the activation of Raf/MEK/ERK cascade upon the treatment with mTOR inhibitor in patients with metastatic cancers as well as in cancer cell lines and prostate cancer animal model, which strongly suggests feedback regulation loops in and crosstalk between the Raf/MEK/ERK and PI3K/Akt cascades [9]. This phenomenon may contribute to drug resistance to inhibitors targeting a single cascade. Another elegant study also supported this notion by showing frequent activation of Raf/MEK/ERK and PI3K/Akt cascades in advanced human prostate cancer [10]. More importantly, several elegant studies have demonstrated synergistic effects in triggering cancer cell death by concomitant interruption of these two pathways both in vitro and in vivo, which indicates that more clinically beneficial pharmacotherapies may be obtained by co-targeting these two pathways simultaneously. However, to our knowledge, all the combined targeted therapies use a mixture of two individual inhibitors for the Raf/MEK/ERK and PI3K/Akt pathways and no single small molecule has been reported or developed to inhibit these two pathways simultaneously. The use of dual pathway inhibitors may provide certain advantages over single pathway inhibitors in the following aspects: 1) enhanced potency and reduced risk of drug resistance; 2) reduced toxicity and improved patient compliance. Thus, the design and development of such dual inhibitors would provide the cancer research community with novel chemical tools and potential newer anticancer agents.

### SUMMARY OF THE INVENTION

Molecules containing the thiazolidine-2,4-dione moiety, such as the anti-diabetic thiazolidinedione (TZD) drug troglitazone, have been recently reported to have anticancer activities at least partially through inhibition of the Raf/MEK/ERK signal cascade [11]. During efforts to design and discover novel templates targeting the Raf/MEK/ERK signaling cascade, thiazolidine-2,4-dione derivatives were developed as potential substrate-specific ERK inhibitors. It was surprisingly discovered that the structural extension of benzylidene to alkylidene converted the TZD analogs into dual inhibitors of the Raf/MEK/ERK and the PI3K/Akt signaling pathways. Thus, these compounds, depicted in generic Formula I, are inhibitors of both pathways, i.e. they are dual inhibitors, and represent novel anticancer agents.

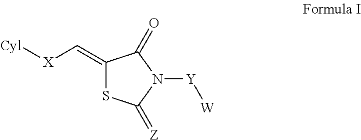

It is an object of this invention to provide a compound of Formula I:

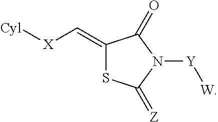

In Formula I, Cyl is selected from the group consisting of: a saturated or unsaturated monocyclic ring with 3-8 carbon atoms which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; admantanyl; phenyl which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; and saturated and unsaturated bi- and tricyclic carbon rings which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; X is C₁-C₄ alkyl; Y is C₁-C₄ alkyl; Z is S or O; and W, where W is i) NR¹R² where R¹ and R² are H or C₁-C₄ alkyl and may be the same or different; or ii) a saturated heterocycle comprising N bonded directly to Y.

In one embodiment, the number of carbon atoms in the saturated monocyclic ring with 3-8 carbon atoms is 3, 4, 5, 6, 7, or 8. In other embodiments, the saturated heterocycle is morpholine, piperidine, piperazine, or pyrrolidine. The compound may be, for example, 3-(2-aminoethyl)-5-(3-cyclohexylpropylidene)-thiazolidine-2,4-dione or 3-(2-aminoethyl)-5-(3-phenylpropylidene)-thiazolidine-2,4-dione.

The invention also provides methods of treating cancer in a patient in need thereof. The method comprises the step of administering to the patient a sufficient quantity of a compound of Formula I:

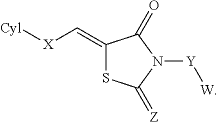

In this formula, Cyl is selected from the group consisting of a saturated or unsaturated monocyclic ring with 3-8 carbon atoms which may be unsubstituted or substituted with one or more substituents selected from the group consisting of C₁-C₄ alkyl, C₁-C₄ alkoxyl, C₁-C₄ alkylcarbonyl, halogen, hydroxyl, amino, nitro, and cyano; admantanyl; phenyl which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; and saturated and unsaturated bi- and tricyclic carbon rings which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; X is C₁-C₄ alkyl; Y is C₁-C₄ alkyl; Z is S or O; and W, where W is i) NR¹R² where R¹ and R² are H or C₁-C₄ alkyl and may be the same or different; or ii) a saturated heterocycle comprising N bonded directly to Y. In one embodiment of the method, the number of carbon atoms in the saturated monocyclic ring with 3-8 carbon atoms is 3, 4, 5, 6, 7, or 8. In other embodiments, the saturated heterocycle is morpholine, piperidine, piperazine, or pyrrolidine. The compound may be, for example, 3-(2-aminoethyl)-5-(3-cyclohexylpropylidene)-thiazolidine-2,4-dione or 3-(2-aminoethyl)-5-(3-phenylpropylidene)-thiazolidine-2,4-dione.

The invention further provides a method of simultaneously inhibiting the Raf/MEK/ERK and PI3K/Akt signaling pathways in a cell. The method comprises the step of exposing the cell to a compound of Formula I:

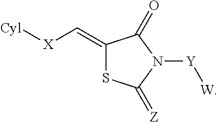

In Formula I, Cyl is selected from the group consisting of: a saturated or unsaturated monocyclic ring with 3-8 carbon atoms which may be unsubstituted or substituted with one or more substituents selected from the group consisting of C₁-C₄ alkyl, C₁-C₄ alkoxyl, C₁-C₄ alkylcarbonyl, halogen, hydroxyl, amino, nitro, and cyano; admantanyl; phenyl which may be unsubstituted or substituted with one or more substituents selected from the group consisting of C₁-C₄ alkyl, C₁-C₄ alkoxyl, C₁-C₄ alkylcarbonyl, halogen, hydroxyl, amino, nitro, and cyano; and saturated and unsaturated bi- and tricyclic carbon rings which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; X is C₁-C₄ alkyl; Y is C₁-C₄ alkyl; Z is S or O; and W, where W is i) NR¹R² where R¹ and R² are H or C₁-C₄ alkyl and may be the same or different; or ii) a saturated heterocycle comprising N bonded directly to Y. In one embodiment of the method, the number of carbon atoms in the saturated monocyclic ring with 3-8 carbon atoms is 3, 4, 5, 6, 7, or 8. In other embodiments, the saturated heterocycle is morpholine, piperidine, piperazine, or pyrrolidine. The compound may be, for example 3-(2-aminoethyl)-5-(3cyclohexylpropylidene)-thiazolidine-2,4-dione or 3-(2-aminoethyl)-5-(3-phenylpropylidene)-thiazolidine-2,4-dione.
In yet another embodiment of the method, the cell that is exposed to the compound is a cancer cell.

The invention also provides a method of inhibiting a kinase enzyme. The method comprises the step of exposing the kinase enzyme to a compound of Formula I:

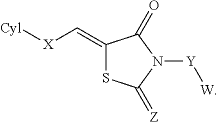

In Formula I, Cyl is selected from the group consisting of a saturated or unsaturated monocyclic ring with 3-8 carbon atoms which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; admantanyl; phenyl which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; and saturated and unsaturated bi- and tricyclic carbon rings which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; X is C₁-C₄ alkyl; Y is C₁-C₄ alkyl; Z is S or O; and W, where W is i) NR¹R² where R¹ and R² are H or C₁-C₄ alkyl and may be the same or different; or ii) a saturated heterocycle comprising N bonded directly to Y. In one embodiment of the method, the number of carbon atoms in the saturated monocyclic ring with 3-8 carbon atoms is 3, 4, 5, 6, 7, or 8. In other embodiments, the saturated heterocycle is morpholine, piperidine, piperazine, or pyrrolidine. The compound may be, for example, 3-(2-aminoethyl)-5-(3-cyclohexylpropylidene)-thiazolidine-2,4-dione or 3-(2-aminoethyl)-5-(3-phenylpropylidene)-thiazolidine-2,4-dione.
In yet another embodiment of the method, the cell that is exposed to the compound is a cancer cell. In some embodiments, the kinase enzyme is MEK1/2, PI3K, CAMK2, CAMK4, AMPK, FLT3, and/or PIM2.

The invention also provides a method of killing or damaging cancer cells. The method comprises the step of exposing the cancer cells to a compound of Formula I:

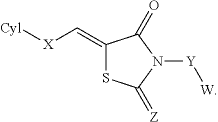

In Formula I, Cyl is selected from the group consisting of: a saturated or unsaturated monocyclic ring with 3-8 carbon atoms which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; admantanyl; phenyl which may be unsubstituted or substituted with one or more substituents selected from the group consisting of C₁-C₄ alkyl, C₁-C₄ alkoxyl, C₁-C₄ alkylcarbonyl, halogen, hydroxyl, amino, nitro, and cyano; and saturated and unsaturated bi- and tricyclic carbon rings which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; X is C₁-C₄ alkyl; Y is C₁-C₄ alkyl; Z is S or O; and W, where W is i) NR¹R² where R¹ and R² are H or C₁-C₄ alkyl and may be the same or different; or ii) a saturated heterocycle comprising N bonded directly to Y. In one embodiment of the method, the number of carbon atoms in the saturated monocyclic ring with 3-8 carbon atoms is 3, 4, 5, 6, 7, or 8. In other embodiments, the saturated heterocycle is morpholine, piperidine, piperazine, or pyrrolidine. The compound may be, for example, 3-(2-aminoethyl)-5-(3-cyclohexylpropylidene)-thiazolidine-2,4-dione or 3-(2-aminoethyl)-5-(3-phenylpropyl idene)-thiazolidine-2,4-dione.
In yet another embodiment of the method, the cell that is exposed to the compound is a cancer cell. In some embodiments, the cancer cells are leukemia, lymphoma, sarcoma, neuroblastoma, lung cancer, skin cancer, head squamous cell carcinoma, neck squamous cell carcinoma, prostate cancer, colon cancer, breast cancer, ovarian cancer, cervical cancer, brain cancer, bladder cancer, and/or pancreatic cancer cells.

### BRIEF DESCRIPTION OF THE DRAWINGS

FIG. 1. U937 cells were treated with Formula II prepared as described in Example 4 [i.e. 3-(2-aminoethyl)-5-(3-cyclohexylpropylidene)thiazolidine-2,4-dione] for 1 h, then stimulated with PMA (200 nM) for 30 min. Lysates were analyzed for p-Akt, p-MEK, p-ERK and α-tubulin by western blot analysis.

FIG. 2. Indicated cancer cells were treated with Formula II synthesized as described in Example 4, 3-(2-aminoethyl)-5-(3-cyclohexylpropylidene)thiazolidine-2,4-dione, at indicated concentrations for 24 hrs, then stained with Annexin-V/PI and analyzed by flow cytometry.

FIG. 3. Effects of Formula II and Formula III on U937 cell cycle. Cells were treated with compounds prepared as described in Examples 4 and 8 [i.e. 3-(2-aminoethyl)-5-(3-cyclohexylpropylidene)thiazolidine-2,4-dione and 3-(2-aminoethyl)-5-(3-phenyl-propylidene)-thiazolidine-2,4-dione, respectfully] for 24 hrs, then cells were stained with PI and analyzed by flow cytometry.

FIG. 4. Treatment with Formula III synthesized as described in Example 8, 3-(2-aminoethyl)-5-(3-phenyl-propylidene)-thiazolidine-2,4-dione, increased % survival of tumor bearing mice. Female B6C3F1 mice (16/group) were injected with 5×10⁵ B16F10 melanoma cells (i.p.), and treatment (50 mg/kg; i.p.) was started 11 days after the tumor cell injection. The moribundity was monitored twice a day until the end of the study. p≧0.05 when compared to vehicle (VH) by Fisher's exact test.

FIG. 5. Treatment with Formula II synthesized as described in Example 4, 3-(2-aminoethyl)-5-(3-cyclohexylpropylidene)thiazolidine-2,4-dione, decreased the lung tumor nodules. Female B6C3F1 mice (10-11/group) were injected with 2×10⁵ B16F10 melanoma cells (i.v.), and treatment with 20 mg/kg dose daily (gavage) was started one day before the tumor cell injection and stopped at day 15 after tumor injection. Mice were sacrificed at day 18 and lungs were removed for counting and observation.

### DETAILED DESCRIPTION

The invention provides compounds of the following Formula I:

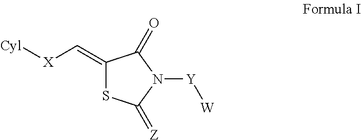

In Formula I, Cyl is (independently from other substituents of the molecule) selected from the group consisting of: saturated and unsaturated monocyclic carbon ring structures containing 3-8 carbon atoms, which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; admantanyl; phenyl which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; and saturated and unsaturated bi- and tricyclic carbon rings which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano;
X is C₁-C₄ alkyl;
Y is C₁-C₄ alkyl;

### Z is O or S; and

W is NR¹R² where R¹ and R² are H or C₁-C₄ alkyl and may be the same or different; or
W is a saturated heterocycle comprising N, the N being bonded directly to Y.

By “saturated heterocycle” we mean a saturated monocyclic carbon ring containing at least one heteroatom atom N as part of the ring. The N atom occupies a position in the ring so that it is bonded directly to Y of the molecule, as depicted in Formula I. The heterocyclic ring is fully saturated (i.e. it does not contain any carbon-carbon double or triple bonds). In addition to N bonded directly to Y, one or more additional positions in the ring(s) may be substituted by other heteroatoms, examples of which include but are not limited to: N, O, S, etc. Exemplary saturated heterocycles that may be used in the practice of the invention include but are not limited to morpholine, piperidine, piperazine, pyrrolidine, etc.

In one embodiment of the invention, the compound of Formula I is the compound 3-(2-aminoethyl)-5-(3-cyclohexylpropylidene)-thiazolidine-2,4-dione shown in Formula II:

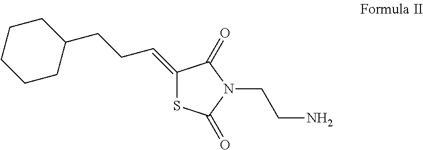

The synthesis of the compound represented by Formula II is described in Example 4.

In another embodiment of the invention, the compound of Formula I is the compound 3-(2-aminoethyl)-5-(3-phenyl-propylidene)-thiazolidine-2,4-dione shown in Formula III:

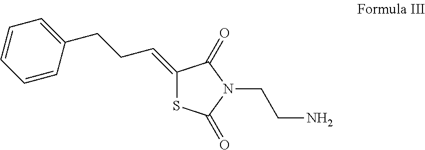

The synthesis of the compound represented by Formula III is described in Example 8.

The invention also provides compositions for the treatment of diseases or conditions associated with the overactivation of ERK1/2, RSK1 and/or Akt. In particular, the invention provides compositions for the treatment of various cancers. The compositions comprise at least one compound of formula (I) and a pharmaceutically acceptable (i.e. a physiologically compatible) carrier, e.g. saline, pH in the range of about 6.5 to about 7.5, and usually about 7.2). Depending on the route of administration, the compositions can take the form of liquids suitable for injection or intravenous administration, aerosols, cachets, capsules, creams, elixirs, emulsions, foams, gels, granules, inhalants, liposomes, lotions, magmas, microemulsion, microparticles, ointments, peroral solids, powders, sprays, syrups, suppositories, suspensions, tablets and tinctures. The amount of the compound of Formula I present in the composition can vary, but us usually in the range of from about 1 to 99%.

The compositions may include one or more pharmaceutically compatible additives or excipients. Commonly used pharmaceutical additives and excipients which can be used as appropriate to formulate the composition for its intended route of administration include but are not limited to:

acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC—CClF₂ and CClF₃);
air displacement agents (examples include but are not limited to nitrogen and argon);
antifungal preservatives (examples include but are not limited to benzoic acid, butylparaben, ethylparaben, methylparaben, propylparaben, sodium benzoate, propionic acids or its salts);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite, tocopherol, vitamin E);
binding materials (examples include but are not limited to block polymers, natural and synthetic rubber, polyacrylates, polyurethanes, silicones and styrene-butadiene copolymers);
buffering agents (examples include but are not limited to potassium metaphosphate, potassium phosphate monobasic, sodium acetate, sodium citrate anhydrous and sodium citrate dihydrate);
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);
chelating agents (examples include but are not limited to edetate disodium and edetic acid); colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel, ferric oxide red, natural colorants such as bixin, norbixin, carmine);
clarifying agents (examples include but are not limited to bentonite);
emulsifying agents (examples include but are not limited to acacia, cetomacrogol, cetyl alcohol, glyceryl monostearate, lecithin, sorbitan monooleate, polyethylene 50 stearate);
encapsulating agents (examples include but are not limited to gelatin and cellulose acetate phthalate);
fillers (examples include but are not limited to sugars, lactose, sucrose, sorbitol, cellulose preparations, calcium phosphates, natural or synthetic gums, solid starch, starch pastes);
flavorants (examples include but are not limited to anise oil, cinnamon oil, cocoa, menthol, orange oil, peppermint oil and vanillin);
humectants (examples include but are not limited to glycerin, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas);
plasticizers (examples include but are not limited to diethyl phthalate and glycerin);
solvents (examples include but are not limited to alcohol, corn oil, cottonseed oil, glycerin, isopropyl alcohol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan monopalmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, fructose, glycerin, mannitol, propylene glycol, saccharin sodium, sorbitol and sucrose);
tablet anti-adherents (examples include but are not limited to magnesium stearate and talc); tablet binders (examples include but are not limited to acacia, alginic acid, carboxymethylcellulose sodium, compressible sugar, ethylcellulose, gelatin, liquid glucose, methylcellulose, povidone and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to carnuba wax and white wax);
thickening agents (examples include but are not limited to beewax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, povidone, sodium alginate and tragacanth); and
wetting agents (examples include but are not limited to heptadecaethylene oxycetanol, lecithins, polyethylene sorbitol monooleate, polyoxyethylene sorbitol monooleate, polyoxyethylene stearate).

Additional additives and excipients suitable for pharmaceutical use such as those described in Remington's The Science and Practice of Pharmacy, 21^{st} Edition (2005), Goodman & Gilman's The Pharmacological Basis of Therapeutics, 11^{th} Edition (2005) and Ansel's Pharmaceutical Dosage Forms and Drug Delivery Systems (8^{th} Edition), edited by Allen et al., Lippincott Williams & Wilkins, (2005) are also considered to be within the scope of the invention.

In one embodiment of the compositions of the invention, one or more (i.e. at least one) additional anti-cancer agent can be added to the composition. Representative anti-cancer agents include, but are not limited to, Erbitux, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel, γ-radiation, alkylating agents including nitrogen mustard such as cyclophosphamide, ifosfamide, trofosfamide, chlorambucil, nitrosoureas such as carmustine (BCNU), and lomustine (CCNU), alkylsulphonates such as busulfan, and treosulfan, triazenes such as dacarbazine, platinum containing compounds such as cisplatin and carboplatin, plant alkaloids including vinca alkaloids, vincristine, vinblastine, vindesine, and vinorelbine, taxoids including paclitaxel, and docetaxol, DNA topoisomerase inhibitors including epipodophyllins such as etoposide, teniposide, topotecan, 9-aminocamptothecin, campto irinotecan, and crisnatol, mitomycins such as mitomycin C, anti-metabolites, including anti-folates such as DHFR inhibitors, methotrexate and trimetrexate, IMP dehydrogenase inhibitors including mycophenolic acid, tiazofurin, ribavirin, EICAR, ribonucleotide reductase inhibitors such as hydroxyurea, deferoxamine, pyrimidine analogs including uracil analogs 5-fluorouracil, floxuridine, doxifluridine, and ratitrexed, cytosine analogs such as cytarabine (ara C), cytosine arabinoside, and fludarabine, purine analogs such as mercaptopurine, thioguanine, hormonal therapies including receptor antagonists, the anti-estrogens tamoxifen, raloxifene and megestrol, LHRH agonists such as goscrclin, and leuprolide acetate, anti-androgens such as flutamide, and bicalutamide, retinoids/deltoids, Vitamin D3 analogs including EB 1089, CB 1093, and KH 1060, photodyamic therapies including vertoporfin (BPD-MA), phthalocyanine, photosensitizer Pc4, Demethoxy-hypocrellin A, (2BA-2-DMHA), cytokines including Interferon, α-Interferon, γ-interferon, tumor necrosis factor, as well as other compounds having anti-tumor activity including isoprenylation inhibitors such as lovastatin, dopaminergic neurotoxins such as 1-methyl-4-phenylpyridinium ion, cell cycle inhibitors such as staurosporine, alsterpaullone, butyrolactone I, Cdk2 inhibitor, Cdk2/Cyclin Inhibitory Peptide I, Cdk2/Cyclin Inhibitory Peptide II, Compound 52 [2-(2-hydroxyethylamino)-6-(3-chloroanilino)-9-isopropylpurine], Indirubin-3′-monoxime, Kenpaullone, Olomoucine, Iso-olomoucine, N⁹-isopropyl-olomoucine, Purvalanol A, Roscovitine, (5)-isomer Roscovitine and WHI-P180 [4-(3′-hydroxyphenyl)amino-6,7-dimethoxyquinazoline, actinomycins such as actinomycin D and dactinomycin, bleomycins such as bleomycin A2, bleomycin B2, and peplomycin, anthracyclines such as daunorubicin, doxorubicin (adriamycin), idarubicin, epirubicin, pirarubicin, zorubicin, and mitoxantrone, MDR inhibitors including verapamil, and Ca²⁺ ATPase inhibitors such as thapsigargin.

In addition, the compounds or compositions of the invention may be administered in conjunction with other health-related and/or cancer treating substances or protocols, including but not limited to: dietary modifications (e.g. vitamin or antioxidant therapy); pain medication or procedures to lessen pain; radiation; various forms of chemotherapy (e.g. administration of platinum drugs, etc.; surgery; cryotherapy; medications to lessen nausea, etc.

The invention also provides methods of treating cancer in a patient in need thereof. The methods comprise a step of administering, to the patient, an effective amount of one or more compounds of formula (I), e.g. as a composition comprising the compound(s). Methods of administration include but are not limited to intradermal, intramuscular, intraperitoneal, intravenous (IV), intratumoral, subcutaneous, intranasal, epidural, oral, sublingual, intranasal, intracerebral, intravaginal, transdermal, rectally, by inhalation, or topically, particularly to the ears, nose, eyes, or skin. Frequently, administration will be IV, although the mode of administration is left to the discretion of the skilled practitioner (e.g. a physician). In most instances, administration will result in the release of a compound of the invention into the bloodstream. However, this need not always be the case, e.g. with topical or intratumoral administration. Further, modes of administration may be combined, e.g. intravenous and intratumoral administration may both be carried out in a patient.

The amount of the compound(s) of Formula I that is administered in one administration is generally in the range of from about 0.1 to about 1 mg/kg of body weight of the patient, and is usually in the range of from about 0.1 to about 1 mg/kg, with a goal of achieving levels of from about 1 to about 5 μM in the blood stream. Those of skill in the art will recognize that administration may be carried out according to any of several protocols, and will generally be determined by a skilled practitioner such as a physician. For example, administration may be once per day, several times per day, or less frequent (e.g. weekly, biweekly, etc.). The amount of the compound that is administered and the frequency of administration may depend on several factors, e.g. the characteristics of the patient (weight, age, gender, overall state of health, etc.); the type and stage of the cancer being treated; the response of the patient to the treatment; etc.

By “an effective amount” we mean an amount that is sufficient to ameliorate, lessen or eliminate symptoms of the disease that is being treated. While in some cases, the patient may be completely “cured” (disease symptoms disappear entirely), this need not always be the case. Those of skill in the art will recognize that substantial benefits may accrue if disease symptoms are only partially mitigated, or if the progress of the disease is slowed. For example, when treating cancer, substantial benefits re quality of life and longevity are obtained by slowing or arresting the growth of a tumor and/or preventing metastasis, etc. even if the tumor itself is not entirely destroyed by exposure to the compounds described herein. In some cases, the cancer cells which are exposed to the compounds of the invention are killed; in other embodiments, the cancer cells are damaged, e.g. prevented from growing or rendered incapable of cell division, etc.

Types of cancer that can be treated using the compounds and methods described herein include but are not limited to: leukemia, lymphoma, sarcoma, neuroblastoma, lung cancer, skin cancer, squamous cell carcinoma of the head and neck, prostate cancer, colon cancer, breast cancer, ovarian cancer, cervical cancer, brain cancer, bladder cancer, pancreatic cancer. The cancer may be at any stage of development, and pre-cancerous cells may also be treated.

The patient or subject that is treated in this manner is usually a mammal, although this is not always the case. Frequently, the mammal is a human, although the methods may also be applied to the treatment of other animals, e.g. in veterinary practice.

The invention also provides methods of simultaneously dual inhibiting the Raf/MEK/ERK and PI3K/Akt signaling pathways in a cell. The methods involve exposing the cells to one or more compounds of the invention, the one or more compounds being present in an amount that is sufficient to inhibit the signaling cascades, usually by at least 50%, in some cases by 60%, 70%, 80%, 90%, 95% or more, or even completely (i.e. 100% inhibition), compared to an untreated control. Those of skill in the art are familiar with methods to measure levels of inhibition of pathways, e.g. by detecting the amount of a metabolite or compound that participates in the pathway or that is made by or in the pathway, e.g. by measuring an amount or degree of mRNA or protein expression, or the amount of protein modification (e.g. phosphorylation or de-phosphorylation), etc. In some cases, the cells in which these pathways are inhibited are cancer cells.

The invention also provides methods of inhibiting one or more kinases (i.e. enzymes with kinase activity) including but not limited to the enzymes MEK1, MEK2, PI3Ka, CAMK2, CAMK4, AMPK, FLT3, and PIM2. As used herein the term “kinase” refers to an enzyme that transfers phosphate groups from high-energy donor molecules, such as ATP, to specific substrates (e.g. other proteins). Kinases are alternatively known as a “phosphotransferases”, and the process is referred to as “phosphorylation”. The methods of the invention involve bringing the enzyme(s) into contact with one or more compounds of the invention, e.g. by contacting, exposing or otherwise providing access of the compound(s) to the enzyme(s). The kinase may be an isolated purified or partically purified enzyme, or may be within a cell (e.g. in a cell cultured in vitro), or within and organism (in vivo). One or more than one (e.g. in some embodiments, all) of the kinases may be inhibited during the practice of the methods. Generally, the activity of the kinase is inhibited by at least about 20, 25, 30, 35, 40, 45, 50, 55, 60, 65, 70, 75, 80, 85, 90, or 95% or even more, e.g. about 100%, compared to a control enzyme which is not exposed to a compound of the invention. Those of skill in the art are familiar with methodology to measure the activity of enzymes, and of kinases in particular. For example, the ability of a kinase to carry out its usual enzymatic activity may be measured, e.g. by detecting a product of that activity, but detecting up- or down-regulation of pathways or components of pathways in which the kinase functions (e.g. measuring levels of phosphorylation of the substrate molecule, mRNA, protein, etc.).

The invention also provides methods of killing or damaging cells exhibiting positive Raf/MEK/ERK and/or PI3K/Akt signaling pathways. By “positive” Raf/MEK/ERK and/or PI3K/Akt signaling pathways we mean overactivation of these two signaling pathways caused by mutation or overexpression of certain proteins within these two signaling pathways. In some embodiments, the cells are cancer cells. The methods involve exposing the cells to one or more compounds of the invention, the one or more compounds being present in an amount that is sufficient to cause the death of the cells, or to cause damage to the cells, e.g. to slow the cells' metabolism, prevent replication, prevent movement, induce apoptosis of the cells, etc. The cells that are killed or damaged may be in vitro or in vivo, i.e. this method may be carried out for clinical purposes (e.g. for the treatment of disease) or in the laboratory (e.g. the compounds of the invention may be used as laboratory reagents.)

Other embodiments of the invention include the treatment of diseases or conditions associated with positive Raf/MEK/ERK and/or PI3K/Akt signaling pathways. These methods comprise the step of administering an effective amount of the compound of formula (I) or a composition thereof to a patient in need thereof to inhibit the Raf/MEK/ERK and PI3K/Akt signaling pathways. Examples of such disease or conditions include but are not limited to cancer, arthritis and other proliferative disease.

The invention is further described by the following non-limiting examples which further illustrate the invention, and are not intended, nor should they be interpreted to, limit the scope of the invention.

### EXAMPLES

Examples 1-16 describe the synthesis of exemplary compounds of the invention.

### Example 1

### Tert-butyl 2-bromoethylcarbamate

To a stirred mixture of 2-bromoethanamine hydrobromide (5.0 g, 24.4 mmol) in 50 mL of anhydrous dioxane was added di-tert-butyl dicarbonate (5.85 g, 26.8 mmol) and triethylamine (3.4 mL, 24.4 mmol) in 25 mL, of dioxane at 0° C. The mixture was then stirred at room temperature for 48 h and filtered to remove the precipitate. The filtrate was condensed and to the residues was added 100 mL of dichloromethane (DCM). The organic phase was washed in turn with 0.5 N HCl, saturated NaHCO₃ and brine and dried over anhydrous Na₂SO₄. Tert-butyl 2-bromoethylcarbamate was obtained as colorless oil after removing the solvents. Yield: 89%. ¹H-NMR (300 MHz, CDCl₃): 3.55-3.52 (t, 2H), 3.48-3.44 (t, 2H), 1.45 (s, 9H).

### Example 2

### Tert-butyl 2-(2,4-dioxothiazolidin-3-yl)ethylcarbamate

To a 500 mL of flask charged with Thiazolidine-2,4-dione (2.0 g, 17.1 mmol), K₂CO₃ (10.6 g, 1.2 e.q), tetrabutylammonium iodide (TBAI, 2.5 g, 0.1 e.q) and 300 mL dry ketone was added tert-butyl-2-bromoethylcarbamate (11.0 mL, 1.5 e.q). The mixture was then refluxed for 10 h and filtered and evaporated to obtain yellow oil, which was added 100 mL, of DCM and then washed with brine and dried over anhydrous Na₂SO₄. The crude product was purified by flash chromatography (hexane/EtOAc: 8/2) to obtain tert-butyl-2-(2,4-dioxothiazolidin-3-yl)-ethylcarbamate in white crystal. Yield: 80%. ¹H-NMR (300 MHz, CDCl₃): δ3.96 (s, 2H), 3.76 (t, 2H), 3.39 (t, 2H), 1.43 (s, 9H); ¹³C-NMR (75 MHz, CDCl₃): δ173.2, 171.3, 167.4 79.2, 41.6, 37.9, 33.3, 27.9.

### Example 3

### 3-Cyclohexylpropioaldehyde

Neat DMSO (1.0 mL, 14 mmol) was added dropwise to a stirred solution of oxalyl chloride (440 uL, 5.0 mmol) in anhydrous DCM (20 mL) at −78° C. under N₂ atmosphere. After 15 min 3-cyclopropanol (610 μL, 4.0 mmol) was slowly added while the temperature was maintained at −78° C. The solution was stirred for 1 h, during which the solution became cloudy. Triethylamine (5.0 mL) was added to the solution and the solution was warmed to room temperature slowly. Water (20 mL) was added and the layers were separated. The aqueous layer was extracted with DCM (3×20 mL). The crude mixture was purified by flash chromatography (EtOAc/hexane=1/10). Yield: 89%. ¹H-NMR (400 MHz, CDCl3): δ9.77-9.76 (t, 1H, J=1.92 Hz), 2.45-2.41 (dt, 2H, J=7.52, 1.92 Hz), 1.71-1.55 (m, 5H), 1.51-1.49 (q, 2H), 1.26-1.11 (m, 4H), 0.93-0.86 (m, 2H); ¹³C-NMR (100 MHz, CDCl3): 203.1, 63.4, 41.5, 37.5, 37.2, 33.4, 33.0, 30.1, 29.3, 26.7, 26.4, 26.2.

### Example 4

### 3-(2-aminoethyl)-5-(3-cyclohexylpropylidene)thiazolidine-2,4-dione

To a mixture of tert-butyl 2-(2,4-dioxothiazolidin-3-yl)ethylcarbamate (260 mg, 1.0 mmol) and cyclohexylpropionaldehyde (140 mg, 1.0 mmol) in 15 mL of anhydrous ethanol was added piperidine (25.5 mg, 0.3 mmol). The clear solution was heated and refluxed overnight and detected with thin layer chromatography (hexane/EtOAc, 8/2). Remove the solvent under vacuum and the residues was purified by flash chromatography (hexane/EtOAc, 8/2) to obtain compound (Z)-tert-butyl 2-(2,4-dioxo-5-(3-cyclohexylpropylidene)tetrahydrothiophen-3-yl)ethylcarbamate as off-white solid. 100 mg of Boc protected (Z)-tert-butyl 2-(2,4-dioxo-5-(3-phenylpropylidene)tetrahydrothiophen-3-yl)ethylcarbamate was dissolved in 4.0 mL of anhydrous EtOAc, and to the solution was added 1.0 mL of HCl solution (4 M in dioxane). The clear solution was stirred and reaction was monitored by TLC. Filter and wash the solid in turn with anhydrous EtOAc and ether to obtain (Z)-3-(2-aminoethyl)-5-(3-cyclohexylpropylidene)thiazolidine-2,4-dione as hydrochloride salt. Yield: 89%. ¹H-NMR (300 MHz, DMSO-d₆): δ8.25 (brs, 3H), 7.29 (m, 5H), 7.03 (t, 1H, J=7.5 Hz), 3.85 (t, 2H, J=6.0 Hz), 3.00 (m, 2H), 2.85 (t, 2H, J=7.5 Hz), 2.54 (q, 2H, J=6.9 Hz); ¹³C-NMR (75 MHz, DMSO-d₆): δ168.3, 165.3, 141.0, 137.9, 129.2, 129.1.9, 127.0, 126.0, 37.2, 33.8, 33.5.

### Example 5

### 3-(2-aminoethyl)-5-(3-cyclopentylpropylidene)thiazolidine-2,4-dione

The title compound was synthesized following the procedure of Example 4, except that cyclopentylpropionaldehyde (126.2 mg, 1.0 mmol) was used as the starting material. ¹H NMR (400 MHz, DMSO-d₆): 8.10-8.00 (m, 3H), 7.05 (t, J=7.7 Hz, 1H), 3.86-3.83 (m, 2H), 3.03 (m, 2H), 2.27-2.21 (q, J=7.5 Hz, 2H), 1.79-1.73 (m, 3H), 1.60-1.47 (m, 6H), 1.09 (m, 2H); ¹³C NMR (100 MHz, DMSO-d₆): 167.5, 164.5, 138.2, 124.8, 39.0, 38.8, 36.4, 33.5, 31.8, 30.5, 24.7.

### Example 6

### 3-(2-aminoethyl)-5-(3-admantanylpropylidene)thiazolidine-2,4-dione

The title compound was synthesized following the procedure of Example 4 except that admantanylpropionaldehyde (98.14 mg, 1.0 mmol) was used as the starting material. ¹H NMR (400 MHz, DMSO-d₆): 8.15 (s, 3H), 7.05 (t, J=7.7 Hz, 1H), 3.04-3.00 (m, 2H), 2.20-2.14 (q, J=7.4 Hz, 2H), 1.94 (brs, 3H), 1.69-1.59 (m, 6H), 1.47-1.46 (d, J=2.3 Hz, 6H), 1.27-1.23 (m, 2H), ¹³C NMR (100 MHz, DMSO-d₆): 167.5, 164.5, 139.1, 124.3, 41.4, 36.5, 36.4, 31.8, 27.9, 25.0.

### Example 7

### 3-(2-aminoethyl)-5-(3-cyclopropylpropylidene)thiazolidine-2,4-dione

The title compound was synthesized following the procedure of Example 4, except that cyclopropylpropionaldehyde (192.3 mg, 1.0 mmol) was used as the starting material. ¹H NMR (400 MHz, DMSO-dₒ): 8.13 (brs, 3H), 7.06 (t, J=7.7 Hz, 1H), 3.83 (t, J=6.0 Hz, 2H), 3.00 (m, 2H), 2.32-2.26 (q, J=7.4 Hz, 2H), 1.43-1.37 (q, J=7.1 Hz, 2H), 0.72-0.68 (m, 1H), 0.42-0.38 (m, 2H), 0.06-0.03 (m, 2H); ¹³C NMR (100 MHz, DMSO-d₆): 167.6, 164.6, 138.1, 124.9, 36.6, 32.2, 31.6, 10.4, 4.5.

### General Procedure for the Preparation of (Z)-3-(2-aminoethyl)-5-(3-substituted-phenylpropylidene)thiazolidine-2,4-dione for Examples 8-16

To a solution of Meldrum's acid derivative (0.5 mmol, synthesized from various aldehyde following the reported procedure of Org. Lett. 2007, 9, 4259-4261), molybdenum hexacarbonyl (6.6 mg, 5 mol %) and N-methylmorpholine-N-oxide (5.9 mg, 10 mol %) in THF (3.0 mL) was added phenylsilane (185 μL, 1.5 mmol). The resulting solution was stirred under an atmosphere of nitrogen at 80° C. for 16 h. After cooling to room temperature water (0.5 mL) was added and the solution stirred for 15 minutes. The solution was dissolved in ethyl ether (50 mL), then was washed with 1N NaOH (3×50 mL) and brine (2×50 mL). The organic phase was dried over anhydrous Na₂SO₄ and concentrated in vacuo. The crude product was purified by flash column chromatography to give various substituted phenylpropionaldehyde.

Corresponding aldehyde from aforementioned reaction was added to the mixture of tert-butyl 2-(2,4-dioxothiazolidin-3-yl)ethylcarbamate (260 mg, 1.0 mmol) and piperidine (25.5 mg, 0.3 mmol) in 15 mL of anhydrous ethanol. The clear solution was heated and refluxed overnight and detected with TLC (hexane/EtOAc, 8/2). Remove the solvent under vacuum and the residues was purified by flash chromatography (hexane/EtOAc, 8/2) to obtain Boc-protected intermediate as white or off-white solid which was dissolved in 4.0 mL of anhydrous EtOAc, and deprotected with 1.0 mL of HCl solution (4M in dioxane). Filter and wash with anhydrous ether to obtain the products described in Examples 8-16 as hydrochloride salts.

### Example 8

### 3-(2-aminoethyl)-5-(3-phenylpropylidene)thiazolidine-2,4-dione

Yield: 84%. ¹H-NMR (400 MHz, DMSO-d₆): δ8.25 (brs, 3H), 7.34-7.21 (m, 5H), 7.06-7.01 (t, 1H, J=7.50 Hz), 3.87-3.83 (t, 2H, J=6.15 Hz), 3.01-2.99 (m, 2H), 2.87-2.82 (t, 2H, J=7.35), 2.59-2.52 (q, 2H). ¹³C-NMR (100 MHz, DMSO-d₆): 168.3, 165.3, 141.0, 137.9, 129.2, 129.1, 126.9, 126.1, 37.2, 33.8, 33.5. Anal. Calcd. for C₁₄H₁₇ClN₂O₂S Calc. C, 53.75; H, 5.48; N, 8.96. Found: C, 53.24; H, 5.40; N, 8.98.

### Example 9

### 3-(2-aminoethyl)-5-(3-(2-ethoxyphenyl)propylidene)thiazolidine-2,4-dione

Yield: 79%. ¹H-NMR (400 MHz, DMSO-d₆): δ8.05 (brs, 3H), 7.21-7.16 (m, 2H), 7.08-7.05 (t, 1H, J=7.6 Hz), 6.96-6.94 (d, 1H, J=7.7 Hz), 6.89-6.85 (dt, 1H, J=0.96, 7.4 Hz), 4.08-4.02 (q, 2H, J=7.0 Hz), 3.85-3.82 (t, 2H, J=6.0 Hz), 3.01 (m, 2H), 2.81-2.78 (t, 2H, J=7.4 Hz), 2.52-2.50 (m, 2H), 1.40-1.36 (t, 3H, J=7.0 Hz); ¹³C-NMR (100 MHz, DMSO-d₆): 167.6, 164.5, 156.4, 137.7, 129.8, 128.1, 127.8, 125.1, 120.2, 111.5, 63.1, 36.6, 31.4, 28.2, 14.7.

### Example 10

### (Z)-3-(2-aminoethyl)-5-(3-(3-ethoxyphenyl)propylidene)thiazolidine-2,4-dione

Yield: 69%. ¹H-NMR (400 MHz, DMSO-d₆): δ8.06 (brs, 3H), 7.22-7.18 (t, 1H, J=7.9 Hz), 7.04-7.01 (t, 1H, J=7.4 Hz), 6.81 (s, 1H), 6.78-6.75 (m, 2H), 4.03-3.98 (q, 2H, J=7.0 Hz), 3.85-3.82 (t, 2H, J=6.0 Hz), 3.02-2.99 (t, 2H, J=6.0 Hz), 2.82-2.79 (t, 2H, J=7.4 Hz), 2.58-2.52 (m, 2H), 1.33-1.30 (t, 3H, J=7.0 Hz); ¹³C-NMR (100 MHz, DMSO-d₆): 167.6, 164.5, 158.6, 141.8, 137.3, 129.4, 125.2, 120.4, 114.5, 112.1, 62.8, 36.6, 32.9, 32.5, 14.6.

### Example 11

### 3-(2-aminoethyl)-5-(3-(4-methoxyphenyl)propylidene)thiazolidine-2,4-dione

Yield: 68%. ¹H-NMR (400 MHz, DMSO-d₆): δ8.04 (brs, 3H), 7.11-7.08 (d, 2H, J=8.6 Hz), 6.96-6.92 (t, 1H, J=7.4 Hz), 6.80-6.78 (d, 2H, J=8.6 Hz), 6.78-6.75 (m, 2H), 3.78-3.75 (t, 2H, J=6.0 Hz), 3.67 (s, 3H), 2.95-2.92 (t, 2H, J=6.0 Hz), 2.72-2.69 (t, 2H, J=7.4 Hz), 2.47-2.41 (m, 2H); ¹³C-NMR (100 MHz, DMSO-d₆): 167.6, 164.5, 157.7, 137.4, 132.1, 129.2, 125.2, 113.8, 54.9, 36.6, 32.9, 32.1.

### Example 12

### 3-(2-aminoethyl)-5-(3-(4-ethoxyphenyl)propylidene)thiazolidine-2,4-dione

Yield: 74%. ¹H-NMR (400 MHz, DMSO-d₆): δ8.05 (brs, 3H), 7.09-7.07 (d, 2H, J=8.6 Hz), 6.96-6.92 (t, 1H, J=7.4 Hz), 6.79-6.76 (d, 2H, J=8.6 Hz), 3.93-3.89 (q, 2H, J=7.0 Hz), 3.78-3.75 (t, 2H, J=6.0 Hz), 2.93 (m, 2H, J=6.0 Hz), 2.71-2.68 (t, 2H, J=7.4 Hz), 2.47-2.41 (m, 2H), 1.40-1.36 (t, 3H, J=7.0 Hz); ¹³C-NMR (100 MHz, DMSO-d₆): 167.6, 164.5, 156.9, 137.4, 132.0, 129.3, 125.2, 114.3, 62.9, 36.6, 32.9, 32.1, 14.6.

### Example 13

### 3-(2-aminoethyl)-5-(3-(4-nitrophenyl)propylidene)thiazolidine-2,4-dione

Yield: 45%. ¹H-NMR (400 MHz, DMSO-d₆): δ8.19-8.17 (d, 2H, J=8.7 Hz), 7.88 (brs, 3H), 7.58-7.56 (d, 2H, Hz), 7.06-7.03 (t, 1H, J=7.5 Hz), 3.83-3.80 (t, 2H, J=5.8 Hz), 3.04-2.99 (m, 4H), 2.65-2.59 (m, 2H); ¹³C-NMR (100 MHz, DMSO-d₆): 167.5, 164.5, 148.7, 146.1, 136.7, 129.7, 125.6, 123.5, 36.8, 32.7, 32.0.

### Example 14

### 3-(2-aminoethyl)-5-(3-(3-nitrophenyl)propylidene)thiazolidine-2,4-dione

Yield: 58%. ¹H-NMR (400 MHz, DMSO-d₆): δ8.18 (s, 1H), 8.11-8.08 (dt, 1H, J=7.8 Hz), 7.87 (brs, 3H), 7.77-7.75 (d, 1H, J=7.8 Hz), 7.64-7.60 (1, 1H, J=7.9 Hz), 7.08-7.04 (t, 1H, J=7.5 Hz), 3.83-3.80 (t, 2H, J=5.8 Hz), 3.03-2.99 (m, 4H), 2.66-2.60 (m, 2H); ¹³C-NMR (100 MHz, DMSO-d₆): 167.6, 164.5, 147.9, 142.7, 136.8, 135.3, 129.9, 125.6, 123.1, 121.3, 36.8, 32.3, 32.2.

### Example 15

### 3-(2-aminoethyl)-5-(3-(4-chlorophenyl)propylidene)thiazolidine-2,4-dione

Yield: 73%. ¹H-NMR (400 MHz, DMSO-d₆): δ7.89 (brs, 3H), 7.37-7.35 (d, 2H, J=7.8 Hz), 7.30-7.28 (d, 2H, J=7.8 Hz), 7.04-7.00 (t, 1H, J=7.5 Hz), 3.83-3.80 (t, 2H, J=5.9 Hz), 3.05-3.02 (t, 2H, J=5.9 Hz), 2.86-2.83 (t, 2H, J=5.9 Hz), 2.58-2.52 (m, 2H); ¹³C-NMR (100 MHz, DMSO-d₆): 167.6, 164.5, 139.3, 137.1, 130.8, 130.2, 128.3, 125.4, 36.8, 32.5, 32.2.

### Example 16

### 3-(2-aminoethyl)-5-[3-(4-butoxyphenyl)-propylidene]-thiazolidine-2,4-dione

Yield 75%. ¹H NMR (400 MHz, DMSO-d₆): 8.08 (brs, 3H), 7.15-7.13 (d, J=8.5 Hz, 2H), 7.01 (t, J=7.4 Hz, 1H), 6.86-6.84 (d, J=8.5 Hz, 2H), 3.92 (t, 6.4 Hz, 2H), 3.83 (t, J=6.0 Hz, 2H), 3.00 (m, 2H), 2.76 (t, J=7.3 Hz, 2H), 2.53-2.48 (m, 2H), 1.69-1.64 (m, 2H), 1.45-1.39 (m, 2H), 0.92 (t, J=7.3 Hz, 3H); ¹³C NMR (100 MHz, DMSO-d₆): 167.5, 164.4, 157.1, 137.3, 131.9, 129.2, 125.1, 114.3, 66.9, 36.5, 32.9, 32.1, 30.7, 18.6, 13.6.

### Example 17

### In Vitro Testing

### Cell Viability Assays

Cells were cultured at a density of 5×10⁴ (U937) or 1×10⁴ (PC-3, DU145, M12, HT29) cells per well in flat bottomed 96-well plates and treated with various concentrations of test compound at 37° C. (5% CO₂). After 24 h, 20 μL of CellTiter 96® Aqueous One Solution Reagent (Promega, Madison, Wis.) was added to each well according to the manufacturer's instructions. After 1 hour, the cell viability was determined by measuring the absorbance at 490 nm using a micro-plate reader.

The results are presented in Table 1. There results show that the Formula II compound inhibited the proliferation of tested cancer cells with an IC₅₀ at single digit micromolar concentrations, with HT29 cells being somewhat less sensitive. The results also demonstrated the activity of Formula III in these cell lines but being less potent that Formula II.

TABLE 1
Inhibition of cancer cell proliferation by compounds synthesized
as described in Examples 4 and 8*

IC₅₀ (μM)

| Cancer Cells | Example 4 | Example 8 |
|---|---|---|
| U937 | 2.21 | 12.23 |
| PC-3 | 4.46 | 14.59 |
| DU145 | 4.80 | 30.83 |
| M12 | 4.51 | 14.81 |
| HT29 | 17.93 | N/A |

*Indicated cells were treated with indicated compounds at various concentrations for 24 hrs, after which cell viability was measured using MTT assay and IC50 was calculated.

### NCI 60 Cell Line Panel Screening

Compound 3-(2-aminoethyl)-5-(3-cyclohexylpropylidene)thiazolidine-2,4-dione was screened at five concentrations in National Cancer Institute (NCI) 60-cell line panel according to NCI's protocol for growth inhibition. The results are presented in Table 2. These results show that Formula II inhibited the proliferation of all 60 cell lines with a single digit micromolar GI₅₀, which indicates that Formula II represents a broad-spectrum anticancer agent.

TABLE 2
NCI 60 cell line panel screening results

| | | Growth Inhibition |
|---|---|---|
| Panel | Cell Line | (GI₅₀, μM) |
| Leukemia | CCRF-CEM | 2.5 |
| Leukemia | HL-60(TB) | 2.04 |
| Leukemia | K-562 | 2.59 |
| Leukemia | MOLT-4 | 2.38 |
| Leukemia | RPMI-8226 | 2.51 |
| Non-Small Cell Lung Cancer | A549/ATCC | 2.5 |
| Non-Small Cell Lung Cancer | EKVX | 2.53 |
| Non-Small Cell Lung Cancer | HOP-62 | 3.25 |
| Non-Small Cell Lung Cancer | HOP-92 | 5.41 |
| Non-Small Cell Lung Cancer | NCI-H226 | 9.98 |
| Non-Small Cell Lung Cancer | NCI-H23 | 3.08 |
| Non-Small Cell Lung Cancer | NCI-H322M | 5.1 |
| Non-Small Cell Lung Cancer | NCI-H460 | 1.95 |
| Non-Small Cell Lung Cancer | NCI-522 | 1.4 |
| Colon Cancer | COLO 205 | 2.19 |
| Colon Cancer | HCC-2998 | 1.78 |
| Colon Cancer | HCT-116 | 1.67 |
| Colon Cancer | HCT-15 | 2.47 |
| Colon Cancer | HT29 | 2.04 |
| Colon Cancer | KM12 | 1.77 |
| Colon Cancer | SW-620 | 1.85 |
| CNS Cancer | SF-268 | 2.23 |
| CNS Cancer | SF-295 | 1.73 |
| CNS Cancer | SF-539 | 1.83 |
| CNS Cancer | SNB-19 | 4.46 |
| CNS Cancer | SNB-75 | 4.56 |
| CNS Cancer | U251 | 1.72 |
| Melanoma | LOX IMVI | 1.4 |
| Melanoma | MALME-3M | 1.8 |
| Melanoma | M14 | 1.68 |
| Melanoma | MDA-MB-435 | 1.85 |
| Melanoma | SK-MEL-2 | 1.9 |
| Melanoma | SK-MEL-28 | 1.77 |
| Melanoma | SK-MEL-5 | 1.7 |
| Melanoma | UACC-257 | 1.69 |
| Melanoma | UACC-62 | 2.14 |
| Ovarian Cancer | IGROV1 | 1.96 |
| Ovarian Cancer | OVCAR-3 | 1.89 |
| Ovarian Cancer | OVCAR-4 | 1.6 |
| Ovarian Cancer | OVCAR-5 | 1.98 |
| Ovarian Cancer | OVCAR-8 | 1.89 |
| Ovarian Cancer | NCI/ADR-RES | 2.2 |
| Ovarian Cancer | SK-OV-3 | 4.04 |
| Renal Cancer | 786-0 | 1.72 |
| Renal Cancer | A498 | 1.57 |
| Renal Cancer | ACHN | 1.84 |
| Renal Cancer | CAKI-1 | 1.64 |
| Renal Cancer | RXF-393 | 2.56 |
| Renal Cancer | SN12C | 2.15 |
| Renal Cancer | TK-10 | 2.01 |
| Renal Cancer | UO-31 | 1.56 |
| Prostate Cancer | PC-3 | 1.91 |
| Prostate Cancer | DU-145 | 1.76 |
| Breast Cancer | MCF7 | 3.13 |
| Breast Cancer | MDA-MB-231/ATCC | 2.08 |
| Breast Cancer | HS 578T | 2.49 |
| Breast Cancer | BT-549 | 2.05 |
| Breast Cancer | T-47D | 2.04 |
| Breast Cancer | MDA-MB-468 | 1.86 |

### Western Blot Analysis

Cells (5×10⁵ per ml) were treated with various concentrations of test compound at 37° C. (5% CO₂) for 3 hrs, then stimulated with TPA at a final concentration of 200 nM for 20 min. Samples from whole-cell pellets were prepared and 30 μg protein for each condition was subjected to SDS-PAGE, transferred onto a PVDF membrane, and blocked with 5% fat-free milk for 30 min. The membrane is probed with primary antibodies overnight at 4° C. followed by incubation with horseradish peroxidase-labeled anti-mouse IgG (1:5000, BD Biosciecne). The immunoreactive bands are detected by chemiluminescence methods (Pierce) and visualized on Kodak Omat film. The following primary antibodies were used: phospho-p44/42 MAPK (ERK1/2, Thr202/Tyr204), p44/42 MAPK, phospho-p90RSK (Thr359/Ser363), RSK1/RSK2/RSK3 (Cell Signaling). Blots were reprobed with antibodies against α-tubulin to ensure equal loading and transfer of proteins.

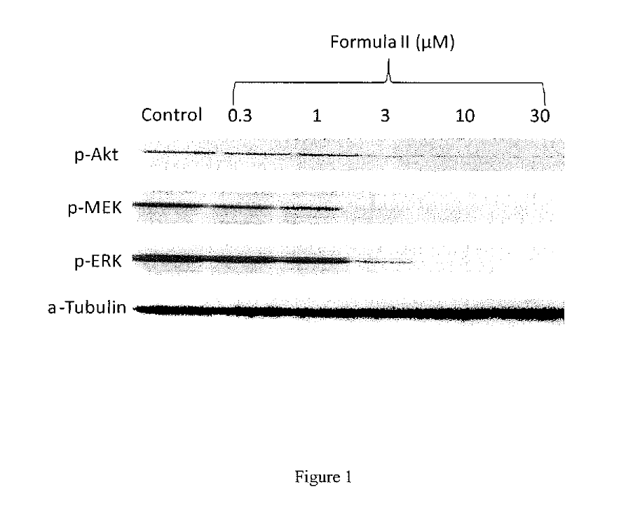

The results are presented in FIG. 1, which shows that Formula II significantly inhibited the phosphorylation of ERK at 3 μM concentrations. However, when the p-MEK level was evaluated, it is notable that Formula II dose-dependently decreased the p-MEK level in U937 cells while treatment with known MEK inhibitor PD184352 resulted in a dose-dependent increase in the p-MEK levels (data not shown), which is consistent with the reported negative feedback mechanism in the Raf/MEK/ERK pathway. This might indicate that Formula II inhibits MEK via a different mechanism. When p-Akt levels were examined, notably, Formula II dose dependently inhibited the phosphorylation of Akt in U937 cells, which clearly indicates that Formula II has specific dual inhibition towards the Raf/MEK/ERK and the PI3K/Akt signaling pathways.

### In Vitro Kinase Screening

Formula II [3-(2-aminoethyl)-5-(3-cyclohexylpropylidene)thiazolidine-2,4-dione] was screened against a panel of kinases at 10 μM concentration by Caliper Life Sciences according to their established protocol. The results are presented in Table 3, and show that Formula II significantly inhibited MEK1, CAMK2, CAMK4 and AMPK at this concentration consistent with the immunoblot studies in U937 cells. Formula II also moderately inhibited PI3Ka. In cell based studies, it was noted that Formula II inhibited the phosphorylation of Akt, a downstream substrate of PI3K, but that inhibition was less potent than the inhibition of p-MEK and p-ERK. Together, these results clearly demonstrate that Formula II inhibits multiple signaling pathways likely to be involved in cancer development simultaneously, suggesting Formula II and its derivatives as novel anticancer agents that target multiple signaling pathways

TABLE 3
In vitro Kinase Selectivity Screening of 3-(2-aminoethyl)-5-(3-
cyclohexylpropylidene)thiazolidine-2,4-dione (Formula II)

| Kinase | % Inhibition |
|---|---|
| ABL | 6 |
| AKT1 | 3 |
| AKT2 | 8 |
| AMPK | 76 |
| AurA | −4 |
| BTK | 12 |
| CAMK2 | 60 |
| CAMk4 | 71 |
| CDK2 | −2 |
| CHK1 | 11 |
| CHK2 | 8 |
| Ck1d | −5 |
| c-Raf | −1 |
| c-TAK1 | 6 |
| DYRK1a | −5 |
| Erk1 | 21 |
| Erk2 | 11 |
| FGFR1 | 3 |
| FLT3 | 38 |
| FYN | 0 |
| GSK3b | 0 |
| HGK | −1 |
| IGF1R | −1 |
| INSR | −2 |
| IRAK4 | −1 |
| KDR | −28 |
| LCK | 4 |
| LYN | 0 |
| MAPKAPK2 | 3 |
| MARK1 | 0 |
| MEK1 | 69 |
| MEK2 | 27 |
| MET | −1 |
| MSK1 | 6 |
| MST2 | −3 |
| p38a | −13 |
| p70S6K | 5 |
| PAK2 | 10 |
| PDK1 | −2 |
| PI3Ka | 45 |
| PIM2 | 38 |
| PKA | −3 |
| PKCb2 | 16 |
| PKCz | −8 |
| PKD2 | 0 |
| PKGa | −2 |
| PRAK | 4 |
| ROCK2 | −1 |
| RSK1 | 2 |
| SGK1 | 14 |
| SRC | 2 |
| SYK | 9 |

### Cell Apoptosis Assays.

Apoptosis was measured by flow cytometry using annexin V/propidium iodide (PI) as staining reagent. Briefly, after treatment with test compound of varying concentrations for varying intervals (4, 8, 18, 36 hrs), cells were washed twice with cold PBS and then resuspended in 1× binding buffer (10 mM HEPES [N-2-hydroxyethylpiperazine-N′-2-ethanesulfonic acid]/NaOH, pH 7.4, 140 mM NaOH, 2.5 mM CaCl2). The cells were then incubated with annexin V-fluorescein isothiocyanate (FITC) (BD PharMingen, San Diego, Calif.) and 5 μg/mL propidium iodide (PI), and incubated for 15 minutes at room temperature in the dark per the manufacturer's instructions. The samples were analyzed by flow cytometry using a Becton Dickinson FACScan (Becton Dickinson, San Jose, Calif.) within 1 hr to determine the percentage of cells displaying annexin V staining (early apoptosis) or both annexin V and PI staining (late apoptosis).

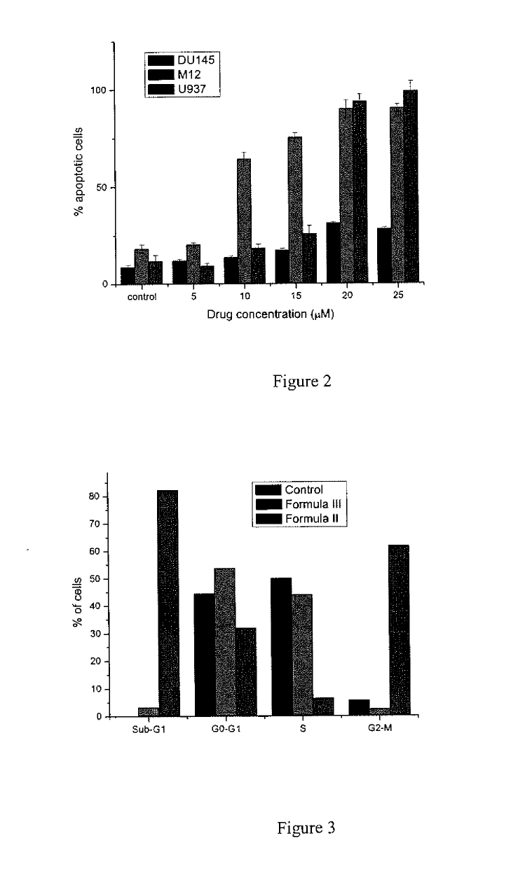

The results are presented in FIG. 2, which shows that Formula II significantly and dose-dependently induced apoptosis in U937 and M12 cells, while exhibiting minimal apoptotic effects in DU145 cells. Since Formula II exhibited similar potency in the inhibition of these three cell lines, this may indicate that the mechanism underlying Formula II's lethal effects in leukemia and prostate cancer cells is cell-specific with apoptotic effects in U937 and M12 cells and necrotic effects in DU145 cells.

### Cell Cycle Analysis.

After treatment of cells with test compound of varying concentrations for 24 hrs, cells were pelleted at 4° C., resuspended, fixed at 4° C. with 67% ethanol overnight, and treated on ice with a PI solution containing 3.8 mM Na citrate, 0.5 mg/mL RNase A (Sigma Chemical Co.), and 0.01 mg/mL PI (Sigma) for 3 hrs. Cell cycle analysis was performed by flow cytometry using Verity Winlist software (Topsham, Me.).

The results are presented in FIG. 3, which shows that treatment of U937 cells with Formula II for 24 h arrested U937 cells at G₂/M phase, an event accompanied by a significant decrease of the S phase population and G0/G1 phase population. However, treatment of U937 cells with Formula III only moderately increased G₀/G₁ population and decreased S population and G2/M population. The differential effects exhibited by Formula II and Formula III in the cell cycle may be due to their different inhibitory potencies on the Raf/MEK/ERK and PI3K/Akt signaling cascades. These results further indicate that Formula II and its derivatives can induce cell death through the interference of the cell cycle, which further confirms the important roles of the Raf/MEK/ERK and PI3K/Akt cascades in cell cycle regulation.

### Example 18

### In Vivo Studies of % Survival of Tumor Bearing Mice

Female B6C3F1 mice (n=16) were injected with 5×10⁵ B16F10 melanoma cells (i.p.), and treatment with Formula III produced as described in Example 8, [3-(2-aminoethyl)-5-(3-phenylpropylidene)thiazolidine-2,4-dione, 50 mg/kg; i.p.] was started 11 days after the tumor cell injection. The moribundity was monitored twice a day until the end of the study.

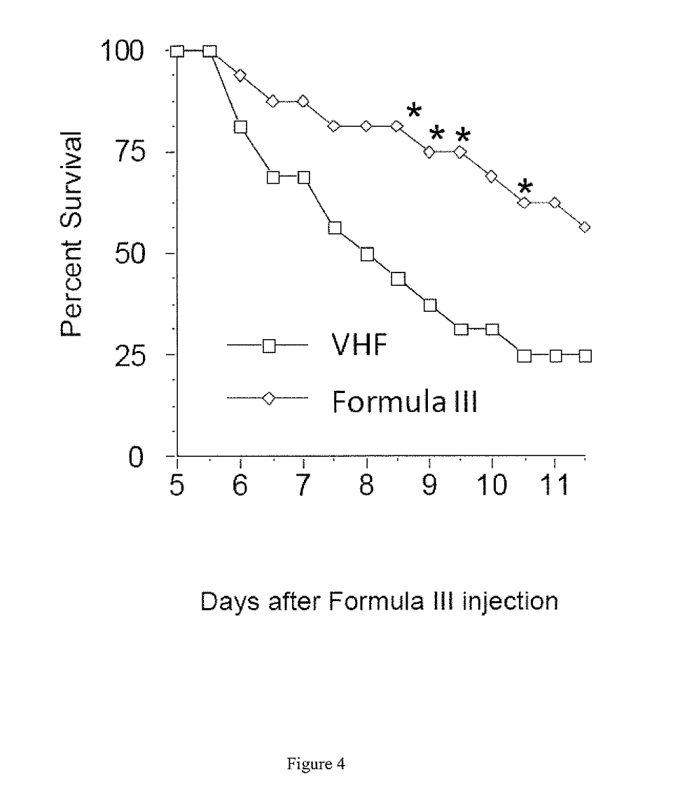

The results are presented in FIG. 4, which shows that Formula III significantly increased the survival rate of B6C3F1 mice bearing B16F10 melanoma cells. These results also demonstrated that Formula III is active in vivo.

### Reduction of Murine B16F10 Melanoma Lung Nodules by 3-(2-aminoethyl)-5-(3-cyclohexylpropylidene)thiazolidine-2,4-dione in Mice

Female B6C3F1 mice (10-11/group) were injected with 2×10⁵ B16F10 melanoma cells (i.v.), and treatment with the compound produced as described in Example 4i.e. 3-(2-aminoethyl)-5-(3-cyclohexylpropylidene)thiazolidine-2,4-dione, 20 mg/kg dosage daily (gavage) was started one day before the tumor cell injection and stopped at day 15 after tumor injection. Mice were sacrificed at day 18 and lungs were removed for counting and observation.

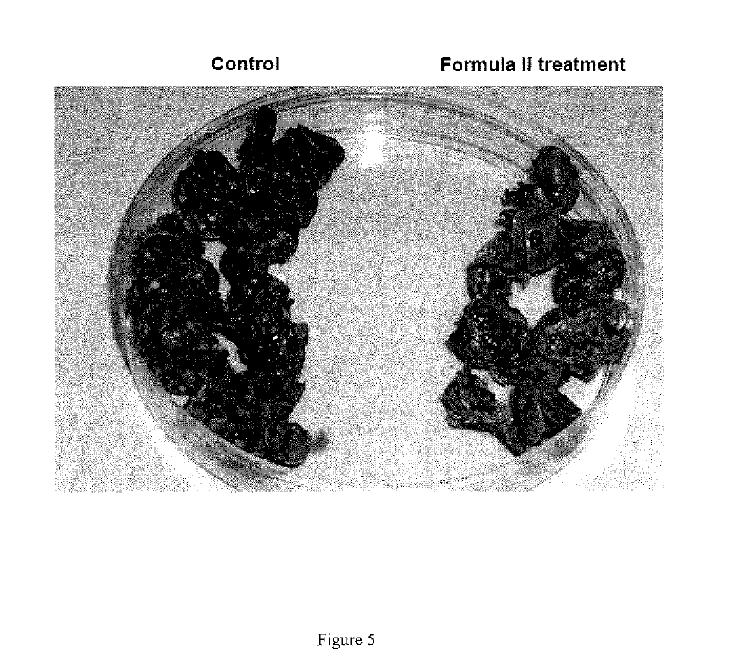

The results are presented in FIG. 5, which shows that Formula II is orally bioavailable and active in vivo in inhibiting the proliferation of B16F10 melanoma cells. These findings are significant for dosage optimization and formulation development in clinical studies.

### Toxicity Studies.

Female B6C3F1 mice (12/group) were given example 4 orally at 30 mg/kg, 50 mg/kg and 80 mg/kg dosage or example 820 mg/kg orally for 21 days. The mice were monitored and no effects on general health and body weight were observed at all three dosages, demonstrating a lack of toxicity in vivo.

Any foregoing applications and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

Citation or identification of any document in this application is not an admission that such document is available as prior art to the present invention.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

1. American Cancer Society. Cancer Facts and Figures 2010.
2. Schubbert, S.; Shannon, K.; Bollag, G. Nat. Rev. Cancer 2007, 7, 295-308.
3. Roberts, P. J.; Der, C. J. Oncogene 2007, 26, 3291-3310.
4. Wong, K. K. Recent Pat Anticancer Drug Discov 2009, 4, 28-35.
5. Hennessy, B. T.; Smith, D. L.; Ram, P. T.; Lu, Y.; Mills, G. B. Nat. Rev. Drug Discov. 2005, 4, 988, 1004.
6. Paz-Ares, L.; Blanco-Aparicio, C.; Garcia-Carbonero, R.; Carnero, A. Clin. Transl. Oncol. 2009, 11, 572-579.
7. Ghayad, S. E.; Cohen, P. A. Recent Pat Anticancer Drug Discov 2010, 5, 29-57.
8. Steelman, L. S.; Abrams, S. L.; Whelan, J.; Bertrand, F. E.; Ludwig, D. E.; Basecke, J.; Libra, M.; Stivala, F.; Milella, M.; Tafuri, A.; Lunghi, P.; Bonati, A.; Martelli, A. M.; McCubrey, J. A. Leukemia 2008, 22, 686-707.
9. Carracedo, A.; Ma, L.; Teruya-Feldstein, J.; Rojo, F.; Salmena, L.; Alimonti, A.; Egia, A.; Sasaki, A. T.; Thomas, G.; Kozma, S. C.; Papa, A.; Nardella, C.; Cantley, L. C.; Baselga, J.; Pandolfi, P. P. J. Clin. Invest. 2008, 118, 3065-3074.
10. Kinkade, C. W.; Castillo-Martin, M.; Puzio-Kuter, A.; Yan, J.; Foster, T. H.; Gao, H.; Sun, Y.; Ouyang, X.; Gerald, W. L.; Cordon-Cardo, C.; Abate-Shen, C. J. Clin. Invest. 2008, 118, 3051-3064.
11. Motomura, W.; Tanno, S.; Takahashi, N.; Nagamine, M.; Fukuda, M.; Kohgo, Y.; Okumura, T. Biochem. Biophys. Res. Commun. 2005, 332, 89-94.

We claim:

1. A compound of Formula I: 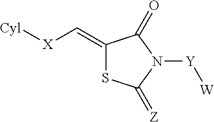
wherein,
Cyl is selected from the group consisting of:
a saturated or unsaturated monocyclic ring with 3-8 carbon atoms which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano;
admantanyl;
phenyl which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; and
saturated and unsaturated bi- and tricyclic carbon rings which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano;
X is C₁-C₄ alkyl;
Y is C₁-C₄ alkyl;
Z is S or O; and
W, where W is
i) NR¹R² where R¹ and R² are H or C₁-C₄ alkyl and may be the same or different; or
ii) a saturated heterocycle comprising N bonded directly to Y.

2. The compound of claim 1, wherein the number of carbon atoms in said saturated monocyclic ring with 3-8 carbon atoms is selected from the group consisting of 3, 4, 5, 6, 7, and 8.

3. The compound of claim 1, wherein said saturated heterocycle is selected from the group consisting of morpholine, piperidine, piperazine, and pyrrolidine.

4. The compound of claim 1, wherein said compound is selected from 3-(2-aminoethyl)-5-(3-cyclohexylpropylidene)-thiazolidine-2,4-dione and 3-(2-aminoethyl)-5-(3-phenyl-propylidene)-thiazolidine-2,4-dione.

5. A method of treating cancer in a patient in need thereof, comprising the step of administering to said patient a sufficient quantity of a compound of Formula I: 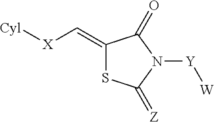
wherein,
Cyl is selected from the group consisting of:
a saturated or unsaturated monocyclic ring with 3-8 carbon atoms which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano;
admantanyl;
phenyl which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; and
saturated and unsaturated bi- and tricyclic carbon rings which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano;
X is C₁-C₄ alkyl;
Y is C₁-C₄ alkyl;
Z is S or O; and
W, where W is
i) NR¹R² where R¹ and R² are H or C₁-C₄ alkyl and may be the same or different; or
ii) a saturated heterocycle comprising N bonded directly to Y.

6. The method of claim 5, wherein the number of carbon atoms in said saturated monocyclic ring with 3-8 carbon atoms is selected from the group consisting of 3, 4, 5, 6, 7, and 8.

7. The method of claim 5, wherein said saturated heterocycle is selected from the group consisting of morpholine, piperidine, piperazine, and pyrrolidine.

8. The method of claim 5, wherein said compound is selected from 3-(2-aminoethyl)-5-(3-cyclohexylpropylidene)-thiazolidine-2,4-dione and 3-(2-aminoethyl)-5-(3-phenyl-propylidene)-thiazolidine-2,4-dione.

9. A method of simultaneously inhibiting Raf/MEK/ERK and PI3K/Akt signaling pathways in a cell, comprising the step of
exposing said cell to a compound of Formula I: 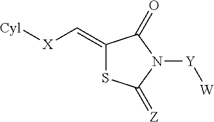
wherein,
Cyl is selected from the group consisting of:
a saturated or unsaturated monocyclic ring with 3-8 carbon atoms which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano;
admantanyl;
phenyl which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; and
saturated and unsaturated bi- and tricyclic carbon rings which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano;
X is C₁-C₄ alkyl;
Y is C₁-C₄ alkyl;
Z is S or O; and
W, where W is
i) NR¹R² where R¹ and R² are H or C₁-C₄ alkyl and may be the same or different; or
ii) a saturated heterocycle comprising N bonded directly to Y.

10. The method of claim 9, wherein the number of carbon atoms in said saturated monocyclic ring with 3-8 carbon atoms is selected from the group consisting of 3, 4, 5, 6, 7, and 8.

11. The method of claim 9, wherein said saturated heterocycle is selected from the group consisting of morpholine, piperidine, piperazine, and pyrrolidine.

12. The method of claim 9, wherein said compound is selected from 3-(2-aminoethyl)-5-(3-cyclohexylpropylidene)-thiazolidine-2,4-dione and 3-(2-aminoethyl)-5-(3-phenyl-propylidene)-thiazolidine-2,4-dione.

13. The method of claim 9, wherein said cell is a cancer cell.

14. A method of inhibiting a kinase enzyme, comprising the step of exposing said kinase enzyme to a compound of Formula I: 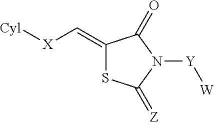
wherein,
Cyl is selected from the group consisting of
a saturated or unsaturated monocyclic ring with 3-8 carbon atoms which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano;
admantanyl;
phenyl which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; and
saturated and unsaturated bi- and tricyclic carbon rings which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano;
X is C₁-C₄ alkyl;
Y is C₁-C₄ alkyl;
Z is S or O; and
W, where W is
i) NR¹R² where R¹ and R² are H or C₁-C₄ alkyl and may be the same or different; or
ii) a saturated heterocycle comprising N bonded directly to Y,
wherein said kinase enzyme is selected from the group consisting of MEK1/2, PI3K, CAMK2, CAMK4, AMPK, FLT3, and PIM2.

15. The method of claim 14, wherein the number of carbon atoms in said saturated monocyclic ring with 3-8 carbon atoms is selected from the group consisting of 3, 4, 5, 6, 7, and 8.

16. The method of claim 14, wherein said saturated heterocycle is selected from the group consisting of morpholine, piperidine, piperazine, and pyrrolidine.

17. The method of claim 14, wherein said compound is selected from 3-(2-aminoethyl)-5-(3-cyclohexylpropylidene)-thiazolidine-2,4-dione and 3-(2-aminoethyl)-5-(3-phenyl-propylidene)-thiazolidine-2,4-dione.

18. A method of killing or damaging cancer cells, comprising the step of exposing said cancer cells to a compound of Formula I: 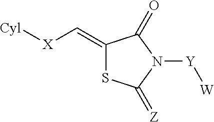
wherein,
Cyl is selected from the group consisting of:
a saturated or unsaturated monocyclic ring with 3-8 carbon atoms which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano;
admantanyl;
phenyl which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano; and
saturated and unsaturated bi- and tricyclic carbon rings which may be unsubstituted or substituted with one or more substituents selected from the group consisting of: C₁-C₄ alkyl, C₁-C₄ alkoxyl, C₁-C₄ alkylcarbonyl, halogen, hydroxyl, amino, nitro, and cyano;
X is C₁-C₄ alkyl;
Y is C₁-C₄ alkyl;
Z is S or O; and
W, where W is
i) NR¹R² where R¹ and R² are H or C₁-C₄ alkyl and may be the same or different; or
ii) a saturated heterocycle comprising N bonded directly to Y,

19. The method of claim 18, wherein the number of carbon atoms in said saturated monocyclic ring with 3-8 carbon atoms is selected from the group consisting of 3, 4, 5, 6, 7, and 8.

20. The method of claim 18, wherein said saturated heterocycle is selected from the group consisting of morpholine, piperidine, piperazine, and pyrrolidine.

21. The method of claim 18, wherein said compound is selected from 3-(2-aminoethyl)-5-(3-cyclohexylpropylidene)-thiazolidine-2,4-dione and 3-(2-aminoethyl)-5-(3-phenyl-propylidene)-thiazolidine-2,4-dione.

22. The method of claim 18, wherein said cancer cells are of a type selected from the group consisting of: leukemia, lymphoma, sarcoma, neuroblastoma, lung cancer, skin cancer, head squamous cell carcinoma, neck squamous cell carcinoma, prostate cancer, colon cancer, breast cancer, ovarian cancer, cervical cancer, brain cancer, bladder cancer, and pancreatic cancer.
